(19) United States
(12) Patent Application Publication
(10) Pub. No.: US 2013/0023969 A1
(43) Pub. Date: Jan. 24, 2013

(54) Cold Pack System and Method

(71) Applicant: Miguel A. Rodriguez, San Antonio, TX (US)

(72) Inventor: Miguel A. Rodriguez, San Antonio, TX (US)

(21) Appl. No.: 13/486,415

(22) Filed: Jun. 1, 2012

Related U.S. Application Data
(60) Provisional application No. 61/492,858, filed on Jun. 3, 2011.

(51) Int. Cl. A61F 7/10; B23P 11/00; B62B 11/00

(52) U.S. Cl. 607/112; 607/114; 280/47.35; 29/428

(57) ABSTRACT

Contiguously attached bags on a substantially cylindrical roll are separated by perforations forming a perforated edge, such that individual bags can efficiently and quickly be filled with ice, removed from the roll, and substantially closed using a sealer that applies an adhesive material about a portion of the bag in an area proximal to its opening.

### CROSS REFERENCE TO RELATED U.S. APPLICATION

This application claims priority to U.S. Provisional Application No. 61/492,858, titled “Cold Pack System and Method,” filed on Jun. 3, 2011.

### FIELD OF INVENTION

The invention relates to care for injuries that require placement of ice on a body part, and provides an efficient, effective, and timely manner for providing ice packs or other cold packs used in an early phase of providing care for such injuries.

### BACKGROUND

Orthopedic injuries to a person's musculoskeletal system happen in a variety of contexts. They occur in athletic activities, entertainment performances, and common workplace situations. People susceptible to various kinds of orthopedic injuries to the musculoskeletal system include, e.g., athletes (either on a team or an individual sport), dancers, workers such as baggage handlers, construction personnel, and military personnel. Sometimes the injury occurs in a professional sport or activity, while other times the injury occurs in an amateur setting. In some instances, the injuries are chronic, while in others the injuries are either acute or sub-acute.

In all such situations, an objective is to provide basic immediate care and emergency care procedures to the injured person. Analgesia, splinting, and control of tissue temperature, for example through the use of cold packs to reduce inflammation and pain, are some techniques for managing injuries in the relatively early immediate care and emergency care stages. The subject matter disclosed herein relates to cold packs.

Frequently, when a person is injured during activities such as those mentioned above, healthcare is provided first at or near the physical location where the injury happened. It may be at a ball field, a performance hall, a construction site, or any of a number of different kinds of locations. People of virtually any age group may find themselves in need of basic assessment and care of injuries during the period of time between when the injury occurs and when the person is transported elsewhere for more formal care or treatment, e.g., an immediate care center and/or hospital emergency room.

A variety of people, either with or without formal medical training, are called upon to provide the assessment and care. Such individuals include athletic trainers, coaches, and volunteers. Increasingly, athletic trainers are providing a form of therapeutic intervention intended to reduce or mitigate the extent of someone's injury. According to occupational data published by the U.S. Department of Labor, in 2008 athletic trainers in this country held approximately 16,300 jobs. By 2018, projected employment of athletic trainers is expected to reach 22,400 in this country. Bureau of Labor Statistics, U.S. Department of Labor, Occupational Outlook Handbook, 2010-11 Edition, Athletic Trainers, available at http://www.bls.gov/oco/ocos294.htm. Accordingly, athletic trainers are part of a continuum of care for the person suffering orthopedic injuries to the musculoskeletal system. The role of the athletic trainer can be particularly prominent during the relatively early stages of the injury condition as well as the rehabilitation period.

A cold pack is routinely used in order to reduce tissue temperature at the injury site, and to control symptoms that frequently accompany orthopedic injuries, e.g., swelling and pain. Often, a cold pack is a bag, vessel, or similar container filled with ice, placed in contact with the person's body on or near the injury site. There are a number of impediments to the efficiency of filling a plastic bag with ice and using the ice-filled bag for any length of time. For example, an inherent difficulty of the bags in some situations is a lack of slip and static electricity, which makes them harder to open. In many situations, a user tears the bag from the roll, or otherwise has a single bag to fill with cold contents. The user might require both hands to hold the bag open, making it cumbersome to handle an ice shovel or other implement with which to put cold contents into the bag.

Also, bags are formed by sealing together at least two pieces of plastic film, which usually requires a seal along one or more edges. Factors such as the location of seals, and the number of seals, may lead to bag leakage. This sometimes occurs at a corner where two edges of a bag meet, where seal clamps touch during production, or where seal clamps are not adjusted properly.

In some situations, numerous bags are linked together and separated by a perforation between any two bags. In most conventional uses, the perforation point is between the bottom of one bag and the top of the next one. Accordingly, another impediment to efficiency is that, when the bag is pulled and separated from the roll, it has a greater tendency to tear at the bottom perforation of the bag. This sometimes leads to bag leakage from the bottom, particularly with the resins and formulations that are used in the majority of bags. Another impediment to efficiency is that, after putting cold contents into a bag, a user typically ties a knot or engages in other manual steps to close the bag.

These problems significantly increase the amount of time and effort needed to fill the bag with ice, and to securely close the opening of the bag through which the ice was placed. Accordingly, there is a substantial need seen in a variety of common activities, from athletic activities to entertainment performances to workplace situations, for a cold pack system and method that increases the efficiency and effectiveness of providing cold therapy to a person. Such a system and method is used and adapted for use in a number of settings, for example training rooms; athletic arenas, stadiums, and fields; fitness centers; performance halls and theatres; ski resorts; immediate care centers and hospital emergency rooms; ambulances; rehabilitation and physical therapy centers; and first aid offices, to name a few.

### BRIEF DESCRIPTION OF THE DRAWINGS

FIG. 1 is a plan view of a cold pack system and method, according to multiple embodiments and alternatives.

FIG. 2 is a plan view of a cold pack system and method, according to multiple embodiments and alternatives.

FIG. 3 is a front elevation view of aspects of a cold pack system and method, according to multiple embodiments and alternatives.

FIG. 4 is a plan view of aspects of a cold pack system and method, according to multiple embodiments and alternatives.

FIG. 5a is a perspective view of a roll base proximal to line a-a in FIG. 5b, according to multiple embodiments and alternatives.

FIG. 5b is a plan view of a cold pack system and method, according to multiple embodiments and alternatives.

FIG. 5c is a plan view of a bag after the teachings herein have been used, according to multiple embodiments and alternatives.

FIG. 6a is a plan view of aspects of a cold pack system and method, according to multiple embodiments and alternatives.

FIG. 6b is a cross-sectional view of a roll base, taken along the same line a-a as in FIG. 5b, according to multiple embodiments and alternatives.

FIG. 7 is a plan view of aspects of a cold pack system and method, according to multiple embodiments and alternatives.

FIG. 8 is a plan view of aspects of a cold pack system and method, according to multiple embodiments and alternatives.

FIG. 9 is a plan view of a cold pack system and method, according to multiple embodiments and alternatives.

FIG. 10 is a plan view of aspects of a cold pack system and method, according to multiple embodiments and alternatives.

FIG. 11 is a front elevation view of aspects of a cold pack system and method, according to multiple embodiments and alternatives.

FIG. 12 is a plan view of aspects of a cold pack system and method, according to multiple embodiments and alternatives.

FIG. 13 is a plan view of aspects of a cold pack system and method, according to multiple embodiments and alternatives.

FIG. 14 is a plan view of aspects of a cold pack system and method, according to multiple embodiments and alternatives.

FIG. 15 is a front elevation view of aspects of a cold pack system and method, according to multiple embodiments and alternatives.

FIG. 16 is a plan view of a cold pack system and method, according to multiple embodiments and alternatives.

FIG. 17a is a plan view of a moving cart as part of a cold pack system and method, according to multiple embodiments and alternatives.

FIG. 17b is a perspective view of the bottom of a moving cart as part of a cold pack system and method, according to multiple embodiments and alternatives.

FIG. 17c is a side elevation view of a moving cart as part of a cold pack system and method, according to multiple embodiments and alternatives.

FIG. 17d is a front elevation view of a moving cart as part of a cold pack system and method, according to multiple embodiments and alternatives.

FIG. 18a is a front elevation view of a moving cart as part of a cold pack system and method, according to multiple embodiments and alternatives.

FIG. 18b is a plan view of a moving cart as part of a cold pack system and method, according to multiple embodiments and alternatives.

FIG. 18c is a side elevation view of a moving cart as part of a cold pack system and method, according to multiple embodiments and alternatives.

FIG. 18d is a perspective view of the bottom of a moving cart as part of a cold pack system and method, according to multiple embodiments and alternatives.

FIG. 18e is a perspective view of a moving cart as part of a cold pack system and method, according to multiple embodiments and alternatives.

The drawings and embodiments described within this specification, including but not limited to in the drawings, are to be understood as illustrative of structures, features and aspects of the present embodiments and not as limiting the scope of the embodiments. It should be understood that the application is not limited to the precise arrangements and instrumentalities shown in the drawing figures, which are not meant to be to scale.

### MULTIPLE EMBODIMENTS AND ALTERNATIVES

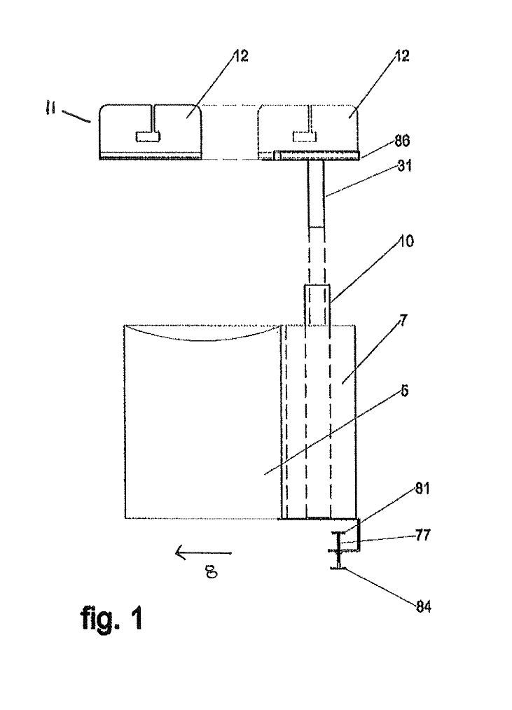

FIG. 1 shows an embodiment of a cold pack system and method, comprising at least one bag 5, a bag roll 7, and a roll base 10 (partially obscured by bag roll 7 as indicated by dashed lines). Bag roll 7 includes a plurality of bags contiguously attached to each other, with any two bags being separated by a perforation running along an edge formed therebetween. Preferably, the bags on the roll are arranged for movement in a first direction (indicated by arrow 8) as each individual bag is pulled away from the roll base.

In some embodiments, a sealer 12 is provided for closing an open end of bag 5 in a relatively secure fashion. In some embodiments, sealer 12 comprises a housing 11 for storing a replaceable supple of adhesive material. Once filled with cold contents, a portion of a bag is then inserted into a slot in the housing in order to seal (i.e., substantially close) the opening of the bag to facilitate retention of the cold contents in the bag.

In some embodiments, sealer 12 is directly or indirectly attached to stem 31, which is of suitable size and dimension to be inserted an opening of roll base 10. As illustrated herein, sealer 12 contacts sealer support 86, which is directly attached to stem 31. In some embodiments, multiple bags are joined together on bag roll 7, such that bag roll 7 has multiple bags that are linked together and rolled through techniques known in the relevant field. In some embodiments, bag roll 7 is in a cylindrical shape, with a substantially hollow area that runs longitudinally at the center of the roll. The bags 5 are formed from suitable material used in the production of ice bags. In some embodiments, the material is a low density polyethylene resin (not claimed) having properties suitable to make the bags light-weight, strong, yet sufficiently pliable and thin to facilitate the efficient placement of bags 5 on bag roll 7. In some embodiments, the properties of the material of bag 5 facilitate both the efficient placement of cold contents into it, as well as the relatively secure closing of bag 5.

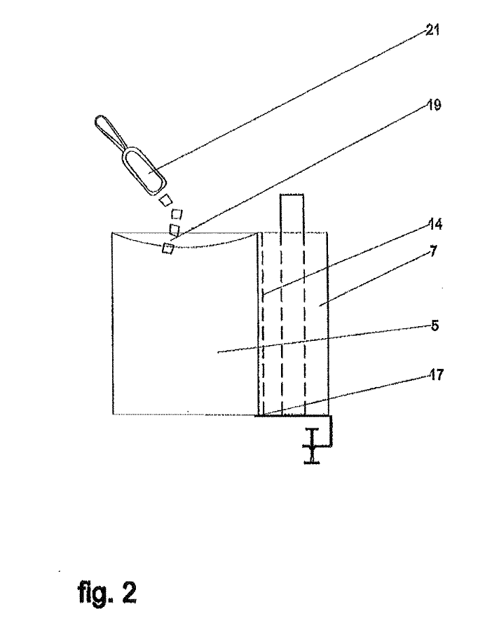

In some situations, it is desirable to have multiple bags joined, yet easily separated. Accordingly, FIG. 2 illustrates embodiments wherein each bag 5 is separated from at least one other bag by a perforated edge 14, which is substantially perpendicular to the direction of movement of the bags along roll 7, as well as substantially perpendicular to the opening of each bag. Also, as shown in FIG. 2, bag roll 7 is placed upon roll base 10, such that a proximal (to roll support 17) end of bag roll 7 contacts the roll support, which is provided in order to limit downward movement. In some embodiments, during use the individual bags are pulled in a first direction away from the roll base, and the roll support is meant to prevent or limit movement in a direction perpendicular to the first direction in order to prevent downward movement of bag roll 7.

As also illustrated in FIG. 2, in some embodiments, each bag 5 has a full or partial opening 19 at one end, for the placement of cold contents (not claimed, e.g., ice or other cold or frozen objects). In some embodiments, bag roll 7 is oriented such that each individual bag 5 opens from the top, rather than from the side. A user places cold contents into bag 5 through bag opening 19, for example using ice shovel 21. Once this occurs, bag 5 is ready to be closed and sealed should a user desire to do so. A user holds together the two ends of bag opening 19 so that opening 19 is substantially closed. In this orientation, bag 5 is ready to be sealed.

In some embodiments, each individual bag 5 is side-sealed (i.e., at the side, rather than being sealed at the bottom). Side-sealing is provided through any of a number of conventional means. An example is where a center-fold plastic sheeting (i.e., film) is fed through sealer clamps, which comprise plates made of durable material like Teflon or steel. Side-sealing refers to an aspect of manufacturing bag 5, prior to the placement of cold contents into the bag. As used elsewhere herein, e.g., in the discussion regarding sealer 12, “sealing” refers to apparatus and steps for closing the opening 19 of bag 5, in order to make it relatively secure against the leakage of cold contents. In that context, sealing is different from the topic of side-sealing.

For side-sealing, through processes known in the art, heat is applied for particular periods of time at particular locations along the film, which causes the film to adhere to itself to form the seal. As this occurs, a perforation is formed between one bag and the next. In some embodiments, bags 5 that are joined together in this way are formed into roll 7 around a bag roll core 16. Accordingly, in some embodiments roll 7 remains in an upright position and has additional support of core 16 to remain in that upright position. (see, e.g., FIG. 2). In this way, opening 19 is at the top of bag 5 rather than at the side, facilitating the work of placing cold contents into the bag. In some embodiments, after cold contents are placed into bag 5, it is separated from roll 7 by tearing along perforation 14.

Many conventional bags are sealed at the bottom, such that the seal is along or near an edge of the bag that is parallel to its opening. By contrast, in some embodiments as described and claimed herein, providing a side-seal proximal to perforated edge 14, rather than along an edge parallel to opening 19, reduces the possibility of cold contents leaking from the bag if they melt.

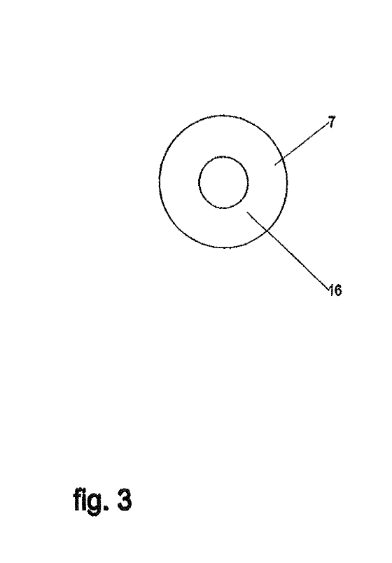

FIG. 3 illustrates bag roll 7 and bag roll core 16 as if viewed from above. In some embodiments, the bags that comprise bag roll 7 are rolled around a bag roll core 16, which is hollow. Bag roll core 16 is formed from materials chosen from the group cardboard, plastic, and metal, any of which is either corrugated or non-corrugated as selectably desired by a user.

In some embodiments, core 16 serves as a base around which bags 5 are rolled, and has a diameter of approximately 3 inches. In some embodiments, core 16 has a substantially planar surface extending slightly beyond the bottom edge of roll 7, to provide additional protection and support for bag roll 7 as it is in an upright position. Core 16 also facilitates shipment, by enabling multiple rolls 7 to be stacked upon each other. Optionally, bag roll core 16 is removed prior to shipment of bag roll 7 for it to be utilized as part of a cold pack system and method. Alternatively, bag roll core 16 is retained within bag roll 7, as illustrated in FIG. 3. Alternatively, bags 5 are formed into a coreless roll without utilizing bag roll core 16.

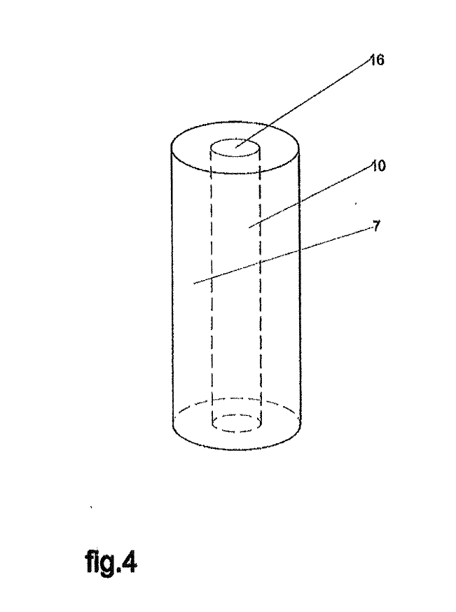

Turning to FIG. 4, in some embodiments, bag roll 7 is placed over roll base 10, wherein base 10 occupies the space within the substantially hollow area of bag roll core 16. In some embodiments, roll base 10 has a longitudinal cross-section that is circular, with a diameter less than, or substantially equal to, the diameter of bag roll core 16. Alternatively, roll base 10 has a cross-section chosen from the group (not shown) triangular, square, pentagonal, hexagonal, and octagonal, in which the diameter of bag roll core 16 is sized to allow bag roll 7 to be slidably placed over roll base 10. In some embodiments, after fitting bag roll 7 over roll base 10, the two are manually separated by lifting bag roll 7 up sufficiently high so that the bottom edge of bag roll 7 clears the top edge of roll base 10.

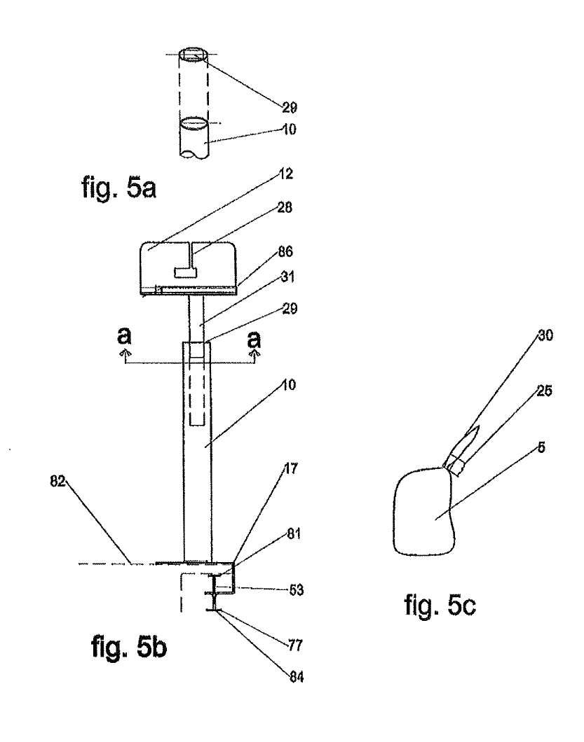

FIGS. 5a-5c illustrate the use of sealer 12 to close bag opening 19 of bag 5 in a relatively secure fashion using an adhesive material, for example one-sided or two-sided tape. FIG. 5a illustrates a section of roll base 10 proximal to line a-a seen in FIG. 5b. In some embodiments, roll base 10 is hollow or partially hollow and has an opening 29 at one end for accommodating stem 31. Stem 31 and sealer support 86 are illustrated in FIG. 5b. In some embodiments, sealer 12 is positioned proximal to bag roll 7. It is sometimes desirable for a user to grasp with one hand a portion of bag 5 directly beneath bag opening 19 (as seen in FIG. 2), and to grasp another portion of bag 5 proximal to cold contents. This forms a neck 30 (see FIG. 5c) that constitutes a relatively narrow section of bag 5, which orients bag 5 for placement into slot 28 of sealer 12 as seen in FIG. 5b. Sealer 12 is then used to wrap around neck 30 a material that keeps bag opening 19 substantially closed and sealed, the results of which are illustrated in FIG. 5c.

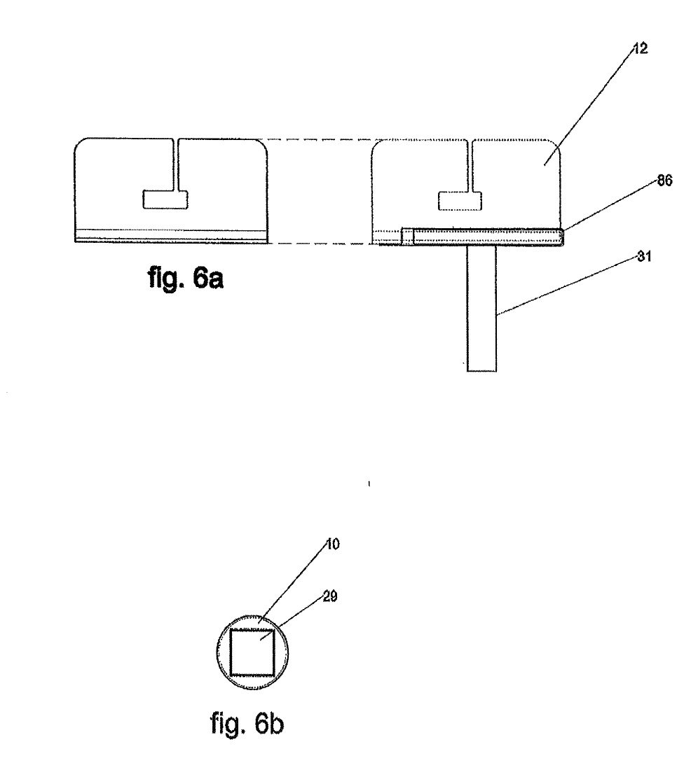

FIG. 6a illustrates sealer 12 in contact with sealer support 86, having stem 31. As illustrated in FIG. 6b, in some embodiments, a base opening 29 of roll base 10 is at least partially hollow to accommodate the insertion of stem 31 into base opening 29. In some embodiments, base opening 29 is rectangular at the cross-section taken along line a-a. Alternatively, the shape of base opening 29 at this cross-section is chosen from the group (not illustrated) triangular, square, pentagonal, hexagonal, and octagonal. Preferably, the shape and dimensions of stem 31 are congruent with those of base opening 29, to facilitate fitting an end of stem 31 into base opening 29 for placement and use of sealer 12 proximal to bag roll 7.

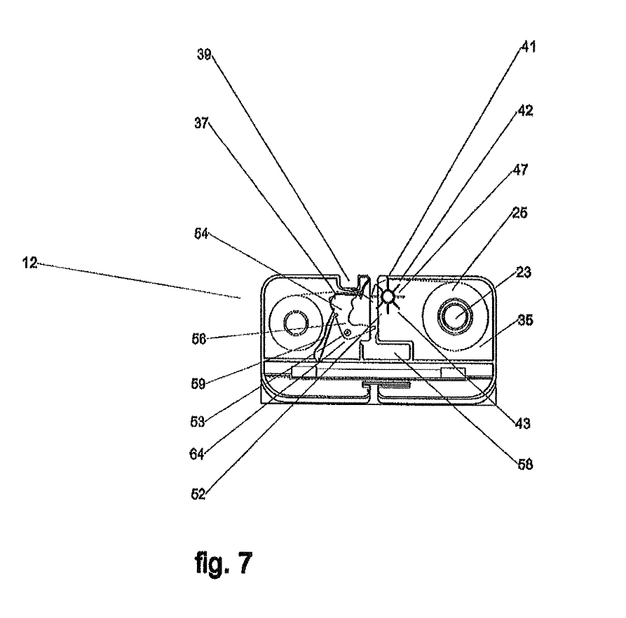

FIG. 7 illustrates the interior of sealer 12. A solid adhesive material 25 is placed on a spool 23. In some embodiments, adhesive material 25 comprises a roll of tape such that adhesive material advancing means create rotational movement about a first axis 35 located relative to spool 23. As it rotates about this axis, adhesive material 25 unspools from spool 23 and travels in a line away from spool 23. Blade 37 is positioned so that, when a portion of adhesive material 25 reaches a pre-determined location distal to spool 23, that portion contacts blade 37 and is sheared from the remainder of the supply of adhesive material 25, and in so doing the cutting urges the adhesive material into engagement with the portion of the individual bag to facilitate closure of the bag and retention of cold contents within the bag. In some embodiments, a guide channel 39 formed in slot 28 controls the path of the adhesive material 25 as it moves linearly away from spool 23, toward blade 37.

In some embodiments, the interior of sealer 12 contains an advancing wheel 41, which comprises at least two projections 42 and 43, separated by a space 47 therebetween. Advancing wheel 41 is situated about a second axis 52 that allows rotation. Advancing wheel 41 rotates in the same direction as the rotation of adhesive material 25 about first axis 35.

In some embodiments, blade 37 is attached to a first arm 54. A second arm 56 is positioned to be in intermittent contact with a distal end (in relation to the center of advancing wheel 41) of a projection 42. As advancing wheel 41 rotates, first arm 54 travels toward the path of adhesive material 25 until it contacts and shears the adhesive material. Once projection 42 rotates a sufficient distance along its path, projection 42 clears the area of second arm 56, bringing the next projection (43) into contact with second arm 56. In this way, it will be seen that, as advancing wheel 41 rotates, projections 42 and 43 also rotate relative to second axis 52, while remaining in the same plane about that axis, similar to how the hands of a clock change positions while remaining in the same axis of the clock's face. In some embodiments, the number of projections exceeds two. Preferably, advancing wheel 41 has from six to nine projections, which are spaced evenly around advancing wheel 41.

As seen in FIG. 7, the area between projections 42, 43 forms space 47, which also moves positions relative to second axis 52. A user applies force to rotate the advancing wheel 41 by inserting the neck 30 of bag 5 into slot 28. In some embodiments, slot 28 is slightly angled opposite the direction of rotation of advancing wheel 41, to facilitate the transmission of force by the user required to move neck 30 through sealer 12. As the user continues to move neck 30 of bag 5 farther into slot 28 apply, the neck moves into space 47. With the application of continued force, the movement of neck 30 causes the positions of projections 42, 43 to change, and causes advancing wheel 41 to rotate. As advancing wheel 41 rotates, it causes adhesive material 25 to travel in a line away from spool 23. In some embodiments, guide channel 39 urges a loop of the adhesive material 25 into space 47, prompting adhesive material 25 to encircle neck 30 of bag 5. A section of adhesive material 25 is sheared as it contacts blade 37, causing that section to remain around neck 30 due to the adhesive. With the application of continued force, neck 30 travels through slot 28 until it reaches center gap 58, at which time bag 5 is removed from sealer 12.

In some embodiments, first arm 54 and second arm 56 are attached and configured to form an angle that resembles an L. In some embodiments, the arms are attached to each other and to a surface of sealer 12 by a screw 53, with other hardware such as a washer (not shown) and a nut (not shown) being optionally included. Screw 53 forms a third axis 64 around which arms 54 and 56 are capable of rotational movement.

In some embodiments, biasing member 59 is joined to second arm 56. In the L-configuration referred to above, biasing member 59 restricts the movement of first arm 54 and establishes a neutral position (as illustrated in FIG. 7) of first arm 54 that is distal to the travel path of adhesive material 25. First arm 54 remains in such neutral position until a user inserts the neck 30 of bag 5 into slot 28 and forces the neck downward, causing advancing wheel 41 to rotate, and pulling an end of adhesive material 25 away from spool 23. Once blade 37, which in some embodiments is attached to first arm 54, contacts and shears the adhesive material 25, biasing member 59 places a compression force on second arm 56, which causes first arm 54 to return to neutral position. In some embodiments, biasing member 59 is a compression spring fixed at a first end to an anchor point within the sealer 12, and at a second end to second arm 56. In some embodiments, first arm 54 also has a blade guard (not illustrated) to shield the fingers or other body parts of a user from blade 37, while the user is accessing the interior of the sealer 12.

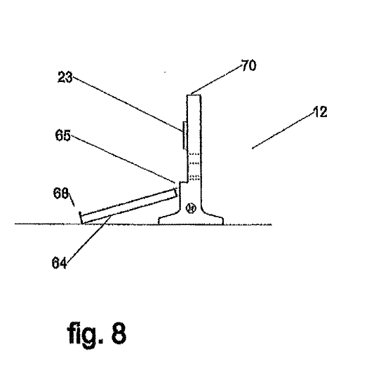

As illustrated in FIG. 8, in some embodiments, the interior of sealer 12 is closed from the exterior by a front plate 64, which is part of housing 11 (see FIG. 1). Optionally, front plate 64 is joined to sealer 12 by at least one hinge 65 as shown in FIG. 8. In this way, it will be seen that the interior of sealer 12 is accessible when front plate 64 is in an opened position, but is not accessible when front plate 64 is in a closed position. In some embodiments, sealer 12 has at least one latch 68 with a corresponding number of latch attachment points 70. These are formed and positioned for matable contact with each latch 68. In some embodiments, front plate 64 of sealer 12 is placed into closed position by engagement of the at least one latch 68 to a like number of latch attachment points 70. FIG. 8 illustrates a first latch 68, which is offset from a first latch attachment point 70, which is in the form of a cutout in the surface of sealer 12 in some embodiments. First latch 68 fits into first latch attachment point 70, thereby releasably securing front plate 64 in the closed position. Alternatively, first latch 68 is a clasp and first latch attachment point 70 is a hook or ring. Alternatively, first latch 68 and first latch attachment are joined by means of hook and loop fasteners. Optionally, a second latch 69 (not illustrated) is matably arranged in like fashion to a second latch attachment point 71 (not illustrated).

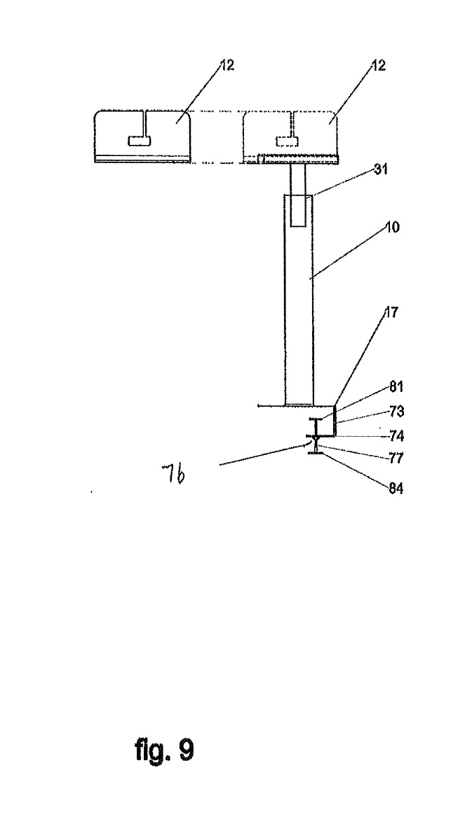

FIG. 9 illustrates roll base 10 attached to roll support 17, through any of several conventional ways selectably chosen by a user. For example, roll base 10 is attached to roll support 17 by a method chosen from the group welding, bolting, gluing, and combinations thereof. In some embodiments, first and second attachment flanges 73, 74 form a bracket that is attached to roll support 17 by a method chosen from the group welding, bolting, gluing, and combinations thereof.

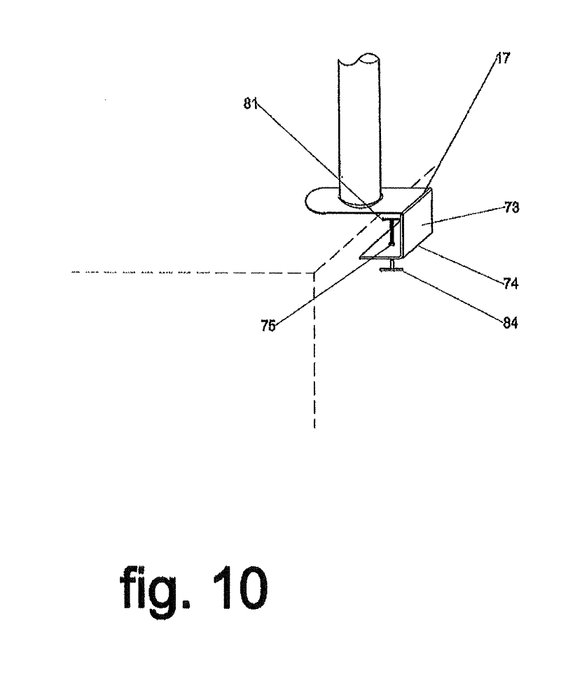

As illustrated in FIG. 10, in some embodiments, roll support 17 is removably joined to a structure 82, which is selectably chosen by a user, e.g., a training table, a countertop, or a moveable cart. FIG. 10 illustrates first attachment flange 73 attached to second attachment flange 74, in which attachment flange 74 and roll support 17 each contacts a surface of structure 82. Alternatively, each attachment flange is attached to roll support 17, and both attachment flanges contact a surface of structure 82, without being attached to the other attachment flange.

Returning briefly to FIG. 9, in some embodiments, a mounting pin 77 has a first end with a planar mounting surface 81 positioned for releasable contact with a surface of structure 82, e.g., the underside of structure 82. In some embodiments, after obtaining releasable contact between planar mounting surface 81 and structure 82, nut 76 is advanced along the threads of mounting pin 77 to hold the mounting pin in relatively static position relative to the structure. As seen in FIG. 10, in some embodiments, mounting pin 77 is inserted through a hole 75 in the surface of second attachment flange 74. Mounting pin 77 comprises a metal cylindrical object of diameter substantially equal to or less than the diameter of hole 75. In some embodiments, mounting pin 77 is partially or fully threaded longitudinally along its cylindrical length. In some embodiments, an end of mounting pin 77 distal to structure 82 comprises a mounting pin handle 84, which allows a user to turn the mounting pin, thereby either advancing or retreating the mounting pin through hole 75, as desired. In some embodiments, mounting pin 77 is oriented substantially perpendicular to roll support 17, so that when planar mounting surface 81 contacts a surface of structure 82, it causes roll support 17 to remain in a substantially stationary position relative to structure 82.

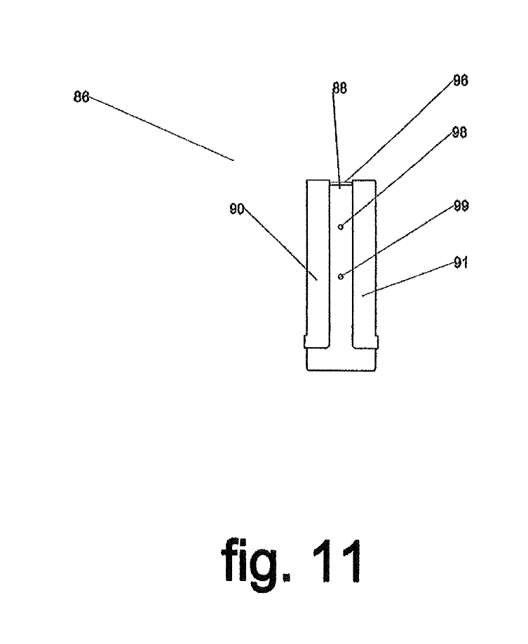

Turning now to FIG. 11, in some embodiments, sealer support 86 comprises a surface 88, a first positioning plate 90, and a second positioning plate 91. Sealer support 86 includes a first hole 98 and, optionally, as illustrated, a second hole 99. Said holes 98, 99 allow sealer support 86 to be releasably joined to structure 82 (not shown in FIG. 11), so that sealer 12 is positioned proximal to its location of use, as selectably desired by a user. In some embodiments, sealer 12 slides along surface 88 of sealer support 86, with the sealer's lateral movement being limited by positioning plates 90, 91. In some embodiments, the forward movement of sealer 12 is limited by a restrictor 96 comprising a positioning plate having a lip formed substantially perpendicular to surface 88. In some embodiments, restrictor 96 comprises a groove in surface 88 into which a protrusion (not illustrated) of sealer 12 fits in order to limit movement.

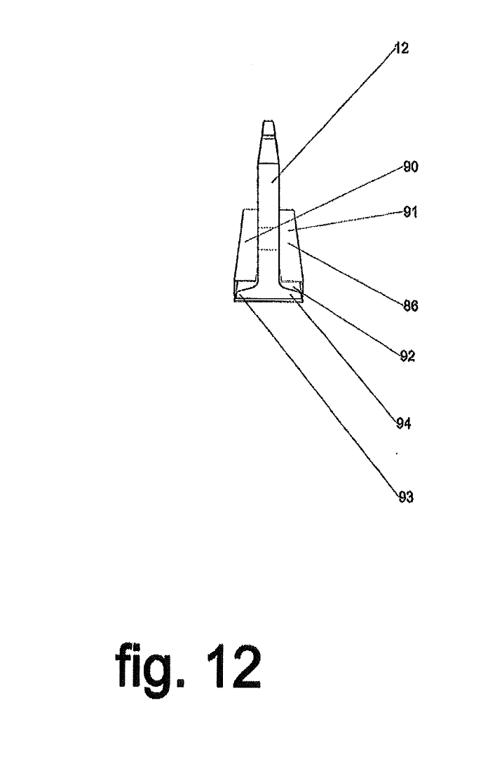

As illustrated in FIG. 12, in some embodiments, first positioning plate 90 and second positioning plate 91 of sealer support 86 contact an edge of said surface 88, and are slightly raised or angled to form an area 92 between the top side of surface 88 and the underside of positioning plates 90, 91. Area 92 is of appropriate shape and dimensions to accommodate a first lateral edge 93 of sealer 12 and a second lateral edge 94 of sealer 12. Optionally, as illustrated in FIG. 12, sealer 12 or sealer support 86 includes stem 31 for relatively secure placement into base opening 29.

It will be noted that the use of bag roll 7 is also optional. In some embodiments, individual bags that were not incorporated as part of a roll are utilized. In such embodiments, bags 5 are stored individually. Optionally, a plurality of bags 5 are linked in succession, with any two bags being linked with a perforated edge 14 between them so that they can be separated. In some embodiments, any two bags 5 that are linked together as part of a coreless roll are linked top-to-bottom, in which the perforation between them is proximal to the top of one bag and the bottom of the adjoining one. Generally, the top of a bag is where opening 19 is found.

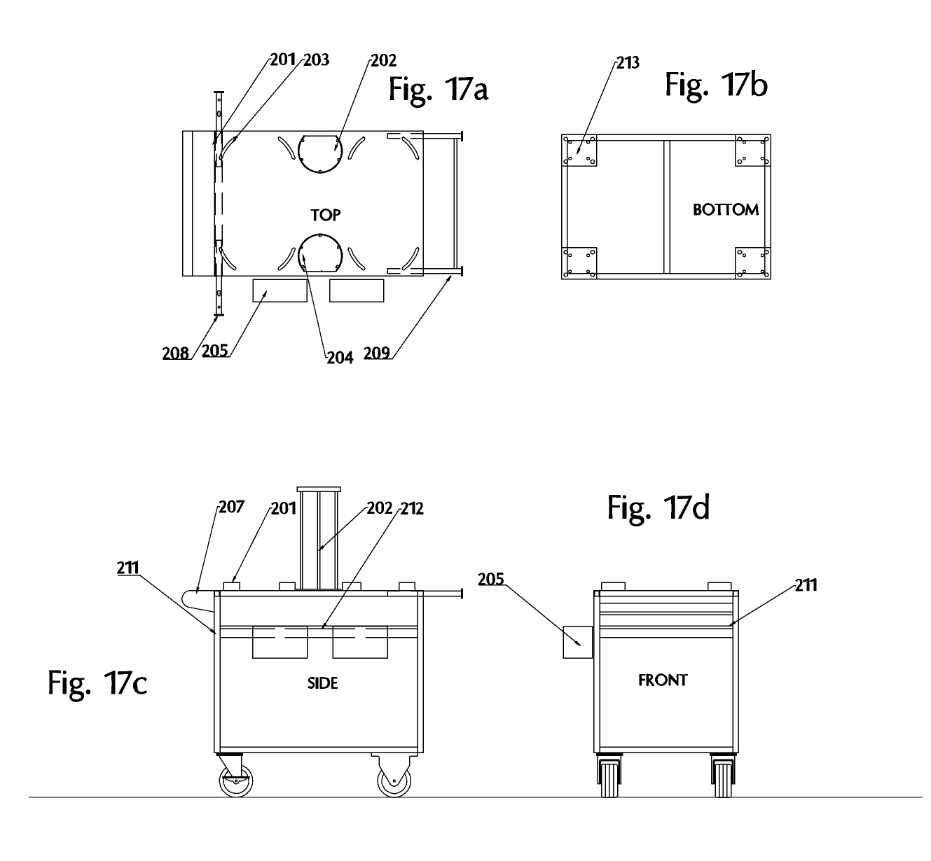

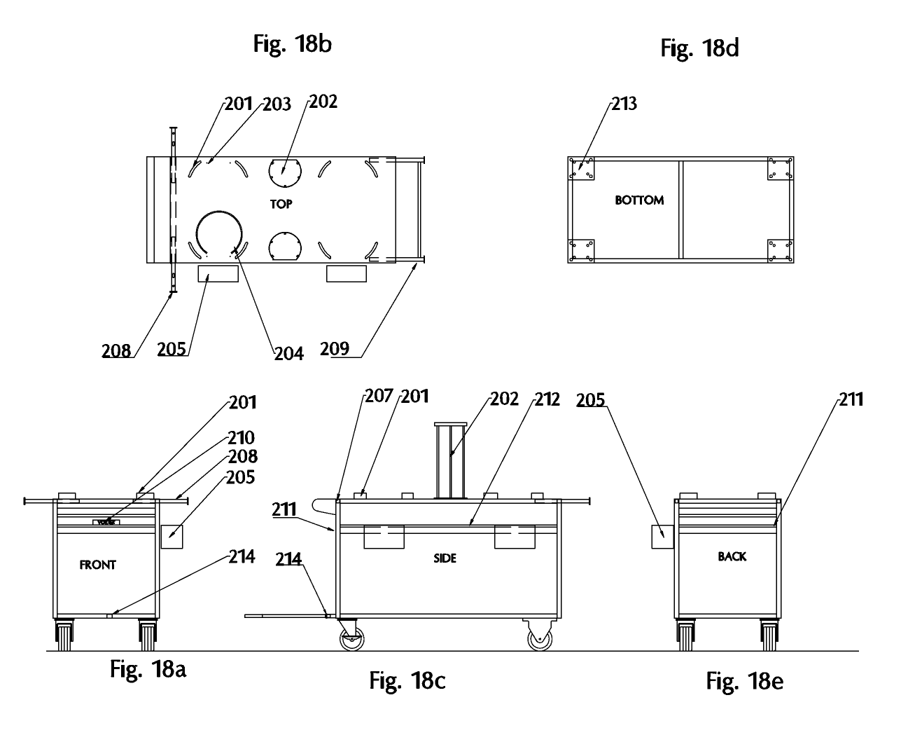

Optionally, one or more features illustrated in FIGS. 1, 3, and/or 8 are arranged on a movable cart (not shown) having a plurality of wheels. Preferably, the cart includes a plurality of caster plates for mounting of casters, similar to that shown in FIGS. 17b and 18d, as discussed below. In some embodiments, features arranged on the movable cart include, but are not limited to, bag roll 7, roll base 10, sealer 12, roll stop 17, and roller 111. If one or more water jugs are transported on the cart, then a plurality of quarter rings or individual rings as described in FIG. 17, discussed below, can be used, or other form of stopper to prevent the jugs from slipping off of the cart. Optionally, the cart includes a top shelf for accommodating accommodating features such as bag roll 7, roll base 10, sealer 12, roll stop 17, and roller 111, and a bottom shelf for accommodating a container for storing cold contents such as an ice chest with a covering that can be in the open or closed position, such as a door (not shown). Preferably, the bottom shelf is angled so the ice chest can be positioned, as desired by a user, in order that gravity will bias the door to the ice chest as either open or closed.

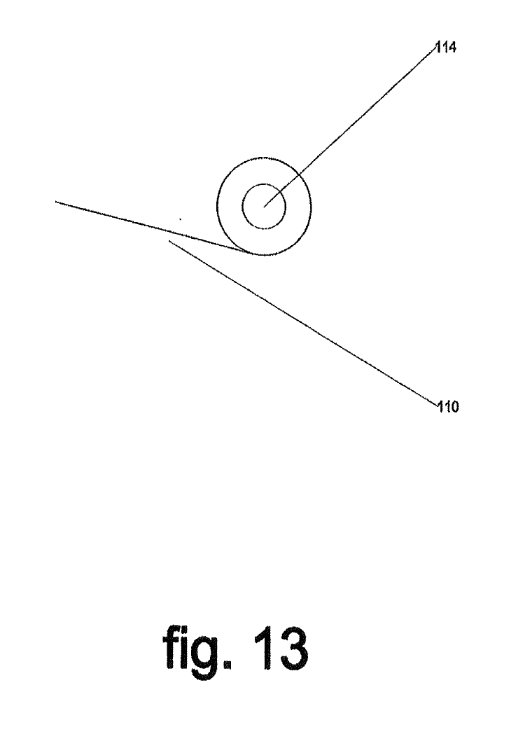

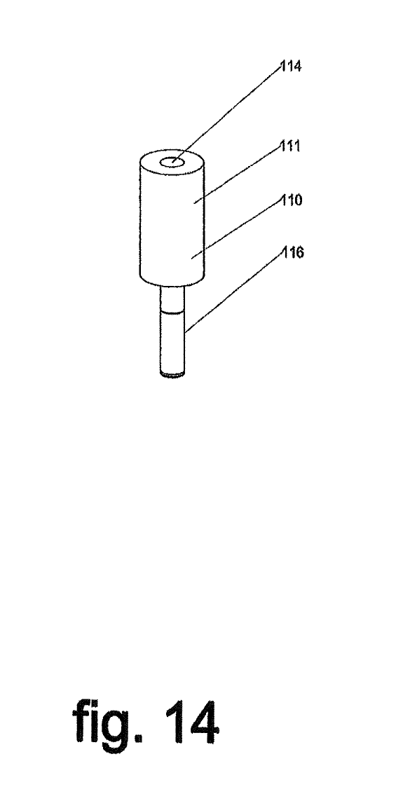

In some embodiments, once bag 5 is filled with cold contents and closed with adhesive material 25, it is helpful to secure the cold pack to a body part of a subject. This is performed with wrap 110, which is illustrated in FIG. 13. Wrap 110 comprises an inner core 114 as shown in FIG. 13, designed to fit snugly around roller 111 with handle 116, as seen in FIG. 14. Wrap 110 is operationally connected to the roller, for example by winding the wrap around the roller core, and then used to securely yet removably place the cold pack on a person's body part.

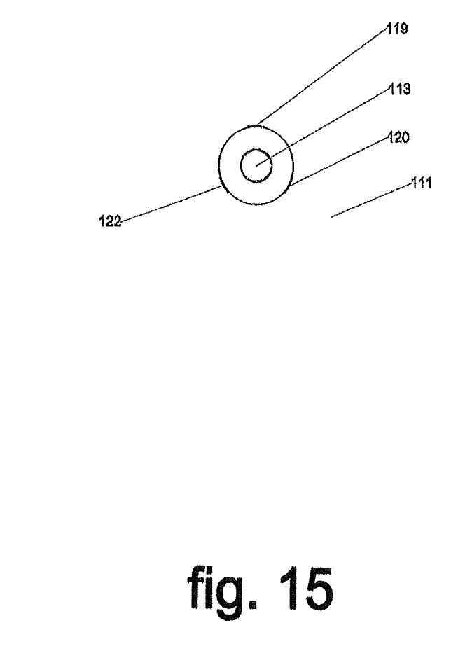

FIG. 15 illustrates roller 111 (without wrap 110) as if viewed from above. Roller 111 comprises a tape roller core 112, and at least one press fit arm 119, which serves to increase the friction between tape roller core 112 and inner core 114 of wrap 110. FIG. 15 illustrates roller 111 with a first press fit arm 119, a second press fit arm 120, and a third press fit arm 121. In some embodiments, tape roller core 112 has a core opening 113 longitudinally along its length. Optionally, tape roller core 112 is solid.

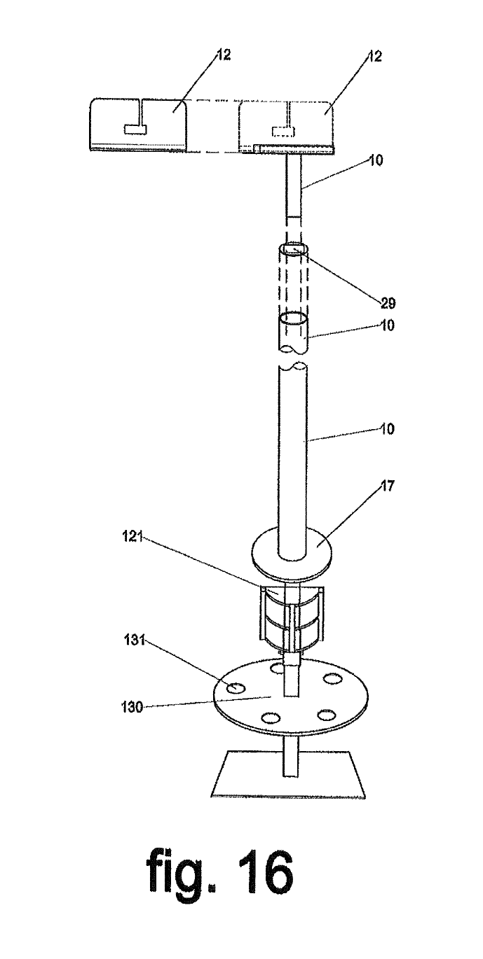

In some embodiments, a pallet 130 is attached to roll base 10, as illustrated in FIG. 16. Pallet 130 includes at least one hole 131 sized for accommodating roller 111 or other similarly sized objects, where those objects can quickly be retrieved and/or replaced after use. In some embodiments, the system includes an ice shovel holder 122 as illustrated in this figure.

Turning now to FIGS. 17a-d and 18a-e, respectively, a movable cart is provided in some embodiments. As best seen in FIG. 17a and FIG. 18b, respectively, a plurality of containment quarter rings 203 are mounted on the top of the cart. Quarter rings 203 are located for the positioning of a jug (not shown), which may be a standard sized jug, so that it will remain stably placed on top of the cart, with minimal sliding. Preferably, four quarter rings 203 are used. In some embodiments, the position of the quarter rings 203 is adjustable, and the position allows the spigot (not shown) of the jug to overhang so that a cup can be positioned under the spigot. This prevents water spillage that is common with other carts. The quarter rings 203 may be placed as selectably desired by a user in order to determine the location of one or more jugs that may be positioned on the cart. In some embodiments, at least one hole (not shown) is drilled in each quarter ring and positioned on the end facing the center of the cart, thus allowing ties (not shown) to be secured to the cart around the jugs for easier transport of the cart and jugs.

In some embodiments, the top of the cart includes a plurality of cup holders 202 as in FIG. 18b, which allow cups to be taken from both sides of cart at the same time. Each cup holder 202 is secured to the top of the cart through conventional means, for example by use of a sufficient number of bolts and a like number of wing nuts. Optionally, the top of the cart includes one or more individual containment rings 204 for holding a smaller jug as shown in FIG. 17a, to allow for the use of one large jug and one small jug at the same time.

As also shown in FIG. 17a and FIG. 18b, one or more drip pans 205 are optionally provided to catch any spillage of water as cups are filled. Optionally, drip pan 205 includes a lip positioned on the back side for releasable engagement with the cart, which allows the drip pan to be removed.

In FIGS. 17b and 18d, respectively, a plurality of caster plates 13, preferably four so that there is one in each corner, allows for mounting of the casters, and easy modification for example by use, as desired, of 4″ casters, 8″ flat free tires or 10″ flat free tires.

As shown in FIGS. 17c and 18c, respectively, at least one pipe 207 may be positioned laterally on the cart, either at an end or at both ends as selectably desired by a user. Preferably, the use of two square pipes 207, one smaller than the other, allows the smaller pipe to slide within the larger pipe and be positioned on both ends of the cart. As desired, a simple screw may be positioned on each small square pipe that fits into a slot (not shown) of the larger pipe to limit movement of the smaller pipe relative to the larger square pipe. In some embodiments, a hole (not shown) is drilled into each small pipe 207 for allowing a holder 208 for sanitation wipes to be releasably engaged with said pipe. In like fashion as the smaller pipe, in some embodiments towel holder 209 is fed into the larger pipe.

As best seen in FIG. 18a, other items may be attached to the cart. For example, a name plate 210 may be affixed to either or both ends of the cart, for example Vortex®. Optionally, a trash can mounting bracket 211 as shown in FIGS. 17c and 18a, respectively, may be attached on the underside of the cart at either side, for allowing a trash can (not shown) to be mounted to the affixed name plate. Optionally, drainage slits 212 for allowing fluid to be removed from a shelf of the cart are provided as shown in FIGS. 17c and 18c. In some embodiments, as shown in FIGS. 18a and 18c, respectively, at least one hitch mount 214 is affixed, which may be permanent, to the end of the football cart which allows the football cart hitch to be attached to the hitch mount and the other end to a vehicle (usually golf cart). This allows the football cart to be pulled by vehicle for ease of movement.

Various features of a cold pack system and method, as disclosed and taught herein, lend themselves to flexibility in the application of these embodiments. It will be readily understood that the embodiments described herein are not limited in their application to the details of the teachings, descriptions, and examples set forth herein. Rather, it will be understood that a cold pack system and method, as described and taught according to multiple embodiments disclosed herein, is capable of other embodiments and of being practiced or of being carried out in various ways by persons having ordinary skill in the art.

Also, it is to be understood that the phraseology and terminology used herein is for the purpose of description and should not be regarded as limiting. The use herein of “including,” “comprising,” “e.g.,” such as,” “for example,” “containing,” or “having” and variations of these words and phrases is meant in a non-limiting way to encompass the items listed thereafter, and equivalents of those, as well as additional items.

Accordingly, the foregoing descriptions are meant to illustrate a number of embodiments and alternatives, rather than to serve as limits on the scope of what has been disclosed herein. The descriptions herein are not intended to be exhaustive, nor are they meant to limit the understanding of the embodiments to the precise forms disclosed.

I claim:

1. A cold pack system for placement of cold contents into individually sealed bags, comprising:
a plurality of contiguously attached bags, each individual bag having an opening and collectively the bags forming a substantially cylindrical roll around a roll base, wherein said bags are arranged on the roll for movement in a first direction relative to the roll base;
a roll support for limiting movement of the roll in a second direction substantially perpendicular to the first direction; and
a sealer comprising a housing having a slot for accommodating a portion of an individual bag; a replaceable supply of adhesive material located substantially within the housing; adhesive material advancing means for positioning a section of adhesive material about a portion of said bag; and a blade substantially within the housing for cutting a section of adhesive material away from the supply of adhesive material, wherein engagement between said section of adhesive material and the portion of said bag substantially closes the opening of said bag.

2. The system of claim 1, further comprising means for attaching the roll support to a planar surface, wherein the planar surface is substantially parallel to the first direction.

3. The system of claim 1, wherein the sealer further comprises a stem with a first end sized to fit within a base opening of the roll base.

4. The system of claim 1, wherein any two of said bags are separated by perforations forming a perforated edge.

5. The system of claim 4, wherein each perforated edge is substantially perpendicular to the opening of said bag.

6. The system of claim 1, wherein the adhesive material comprises tape arranged upon a rotatable spool, the tape being chosen from the group one-sided and two-sided tape.

7. The system of claim 1, wherein the slot is configured to accept a portion of said bag for passage along a path defined by a guide channel terminating at the adhesive material, the terminus of the guide channel being located proximal to the blade, such that a mechanical force applied against the terminus of the guide channel cuts a section of adhesive material away from the supply of adhesive material and urges the adhesive material into closing engagement with the portion of said individual bag, wherein the movement of said bag along the path to the terminus is capable of generating the mechanical force.

8. The system of claim 1, wherein the sealer further comprises a sealer support having a plurality of positioning plates for limiting the motion of the sealer.

9. The system of claim 1, further comprising at least one roller operationally connected to a wrap, wherein the wrap is capable of removably secure placement to a body part.

10. The system of claim 9, further comprising a pallet having at least one hole sized for accommodating said at least one roller.

11. A method for preparing a cold pack for placement on a body part, comprising the steps of:
providing a bag roll comprising a plurality of contiguously attached bags around a roll base, wherein each individual bag has an opening, and said plurality of bags are arranged for movement in a first direction relative to the roll base;
positioning a roll support for limiting movement of the roll in a second direction substantially perpendicular to the first direction;
configuring a replaceable supply of adhesive material within a sealer, the sealer comprising a housing having a slot for accommodating a portion of an individual bag;
providing, within the housing, adhesive material advancing means for positioning a section of adhesive material about a portion of said bag; and
providing a blade for cutting a section of adhesive material away from the supply of adhesive material, wherein engagement between said section of adhesive material and the portion of said bag substantially closes the opening of said bag.

12. The method of claim 11, further comprising providing means for attaching the roll support to a planar surface, wherein the planar surface is substantially parallel to the first direction.

13. The method of claim 11, further comprising forming the sealer with a stem having a first end sized to fit within a base opening of the roll base.

14. The method of claim 13, further comprising forming the sealer with a sealer support having a plurality of positioning plates for limiting the motion of the sealer.

15. The method of claim 11, further comprising providing perforations forming a perforated edge between any two of said bags, wherein each perforated edge is substantially perpendicular to the opening of said bag.

16. The method of claim 11, further comprising configuring the slot to accept a portion of said bag for passage along a path defined by a guide channel terminating at the adhesive material, the terminus of the guide channel being located proximal to the blade, such that a mechanical force applied against the terminus of the guide channel cuts a section of adhesive material away from the supply of adhesive material and urges the adhesive material into closing engagement with the portion of said individual bag, wherein the movement of said bag along the path to the terminus is capable of generating the mechanical force.

17. The method of claim 16, further comprising removing an individual bag from the bag roll by tearing along the perforation, inserting a portion of said bag into the slot, and moving said bag along the path defined by the guide channel to the terminus of the guide channel.

18. The method of claim 11, further comprising providing at least one roller operationally connected to a wrap, wherein the wrap is cable of removably secure placement to a body part.

19. A method for applying a cold pack to an individual, comprising providing a cold pack system according to claim 1; removing an individual bag from the bag roll by tearing along the perforation; inserting a portion of said bag into the slot; moving said bag along a path defined by a guide channel terminating at the adhesive material, wherein the terminus of the guide channel is proximal to the blade; and wrapping the cold pack to a body part of the individual.

20. The system of claim 1, further comprising a movable cart having a plurality of wheels and a first shelf upon which bag roll, roll base, and roll stop 17, and a second shelf capable of accommodating a container for storing cold contents.
